(19) United States
(12) Patent Application Publication
(10) Pub. No.: US 2012/0116176 A1
(43) Pub. Date: May 10, 2012

(54) HANDHELD BOIFEEDBACK DEVICE AND METHOD FOR SELF-REGULATING AT LEAST ONE PHYSIOLOGICAL STATE OF A SUBJECT

(75) Inventors: Christine S. Moravec, Shaker Heights, OH (US); Michael G. McKee, Shaker Heights, OH (US); Dana L. Schneeberger, Cleveland Heights, OH (US)

(73) Assignee: The Cleveland Clinic Foundation

(21) Appl. No.: 13/289,451

(22) Filed: Nov. 4, 2011

Related U.S. Application Data
(60) Provisional application No. 61/410,031, filed on Nov. 4, 2010.

(51) Int. Cl. A61B 5/00

(52) U.S. Cl. 600/300

(57) ABSTRACT

A handheld, portable device for providing biofeedback to a subject includes a housing, at least one sensor, and a controller. The housing has a user interface operably connected thereto. The at least one sensor is configured to sense physiological parameter. The at least one sensor is operably connected to the housing. The controller is configured to provide biofeedback to the subject. The controller is in electrical communication with the at least one sensor.

### RELATED APPLICATION

This application claims the benefit of U.S. Provisional Patent Application Ser. No. 61/410,031, filed Nov. 4, 2010, the entirety of which is hereby incorporated by reference for all purposes.

### FIELD OF THE INVENTION

The present invention relates to a biometric device, and more particularly to a handheld biofeedback device for measuring and/or self-regulating at least one physiological state of a subject.

### BACKGROUND OF THE INVENTION

The measurement and analysis of biological signals and investigation of their correlation with psychological processes has a long history. Early biofeedback devices were relatively simple, with the feedback signal typically being represented by the position of an oscilloscope dot on a screen or the pitch of an audio tone. Advances in the processing and graphical capabilities of home computers meant that, by the early 1980's, the feedback provided to the user could be presented in a much richer context for use both in therapeutic and consumer products.

Recently, the use of biofeedback devices in both clinical and commercial settings has increased, finding widespread application in therapies for anxiety, sleep disorders, and attention-deficit hyperactivity disorder, among others. Several biofeedback products for stress management have also come to market. Reducing the stress associated with modern, urban living is important to the general health of society; hence, these products have a useful role to play in helping people to monitor and enhance their mental and physical well-being.

Traditional biofeedback systems are typically attached to the user via tape or some sort of binding. Further, traditional systems are large, heavy, non-portable wired arrangements that do not provide the user with a rewarding experience or desire for repeated use. Additionally, due to variations in human physiology, biometric signals can be difficult to accurately measure and track across the population, making it difficult to provide useful biofeedback on an individual basis.

### SUMMARY OF THE INVENTION

In accordance with one aspect of the present invention, a handheld, portable device for providing biofeedback to a subject includes a housing, at least one sensor, and a controller. The housing has a user interface operably connected thereto. The at least one sensor is configured to sense physiological parameter. The at least one sensor is operably connected to the housing. The controller is configured to provide biofeedback to the subject. The controller is in electrical communication with the at least one sensor.

In accordance with another aspect of the present invention, a method is provided for self-regulating at least one physiological state of a subject. One step of the method includes providing a portable, handheld device comprising a housing having a user interface operably connected thereto, at least one sensor operably connected to the housing, and a controller that is in electrical communication with the at least one sensor. The device is operated to provide at least one instruction to the subject based on at least one collected and recorded physiological parameter. A decision is then made by the subject whether to take an action in response to the at least one instruction to regulate the at least one physiological state.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 A is a perspective view showing a front side of a handheld, portable device for providing biofeedback to a subject constructed in accordance with one aspect of the present invention;

FIG. 1B is a perspective view showing a back side of the handheld, portable device in FIG. 1A;

FIG. 2A is a perspective view showing a front side of a handheld, portable device for providing biofeedback to a subject constructed in accordance with another aspect of the present invention;

FIG. 2B is a perspective view showing a back side of the handheld, portable device in FIG. 2A;

FIG. 3 is a perspective view showing an alternative configuration of the back side of the device in FIGS. 1A-B;

FIG. 4 is a process flow diagram illustrating a method for self-regulating at least one physiological state of a subject according to another aspect of the present invention; and

FIG. 5 is a perspective view showing two digits of a subject placed on fingertip sensors shown in FIG. 3.

### DETAILED DESCRIPTION

The present invention relates to a biometric device, and more particularly to a handheld biofeedback device for measuring and/or self-regulating at least one physiological state of a subject. Commercially available handheld biofeedback devices are surprisingly popular with consumers as they rely on only one physiological parameter and do not record data. Conversely, the present invention provides multi-parameter feedback, indicators of various physiological states (e.g., stress), and guidance on managing the various physiological states. Advantageously, the present invention provides physiological monitoring and biofeedback training that allows a subject to self-regulate for a variety of different purposes, including enhancing personal health and wellness, alleviating a disease state, and improving performance.

Generally speaking, biofeedback methods and devices involve training processes that allow subjects to facilitate changes in their physiological parameters in the direction of health and wellness. Over time, a subject can be trained with biofeedback methods and devices to exercise greater control over these functions. In contrast to other forms of therapy in which treatment is imposed upon the subject, biofeedback methods and devices allow the subject to gradually integrate the training processes into nearly automatic responses.

The present invention includes several features that enable self-regulation of various physiological states through feedback of physiological monitoring. Examples of such features include highly accurate and rapidly responding physiological sensors, circuitry and software that provide rapid feedback to a subject, a convenient and simple user interface, sensors and circuits validated with well-established physiological measures of autonomic nervous system (ANS) function and electroencephalography (EEG) waves; calibration of biofeedback parameters in simulated stress and performance settings; and a well-organized, electronically-available set of instructions for regulating various physiological states.

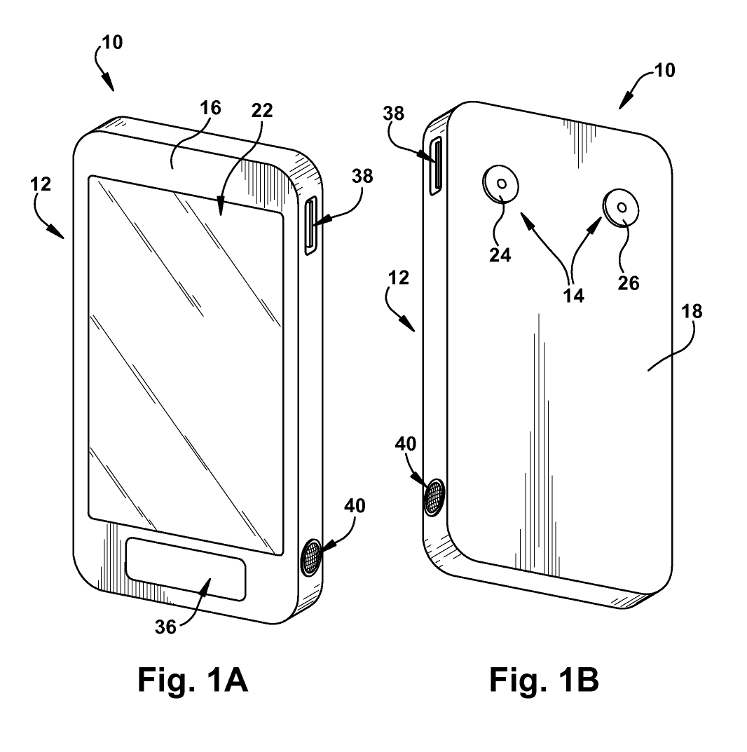

As described in more detail below, the features of the present invention can be individually or collectively incorporated to provide: (1) a portable, handheld biofeedback device 10 (FIGS. 1A-B) that measures and stores different physiological parameters, calculates a biofeedback score (e.g., a stress score) based on the measured parameters, and provides instruction based on the biofeedback score for self-regulating a physiological state of a subject; (2) a remote workstation system that allows a subject to optimize physiology privately while engaged in routine activity (e.g., home or workplace) or high demand/performance situations; and (3) a personally-calibrated wireless sensing system that can be used to optimize performance based on ANS and EEG recordings and bio/neurofeedback. It will be appreciated that these features, as well as the potential for a smaller device 10 (i.e., miniaturization) and wireless remote transmission are included within the scope of the present invention.

The following description relates to exemplary aspects of the present invention in the form of a handheld, portable device 10 that can be used to provide biofeedback to a subject in substantially real-time to promote self-regulation of at least one physiological state of a subject. In addition to the particular methods and devices described below, it should be appreciated that other methods and devices are intended to be within the scope of the present invention. Where alternative configurations and/or aspects of the present invention are not explicitly described, it is not the intention of applicants to limit the present invention to the exact description provided herein. In particular, it should be appreciated that various combinations of features described herein may be incorporated into a single device, and that such device will fall within the scope of the present invention.

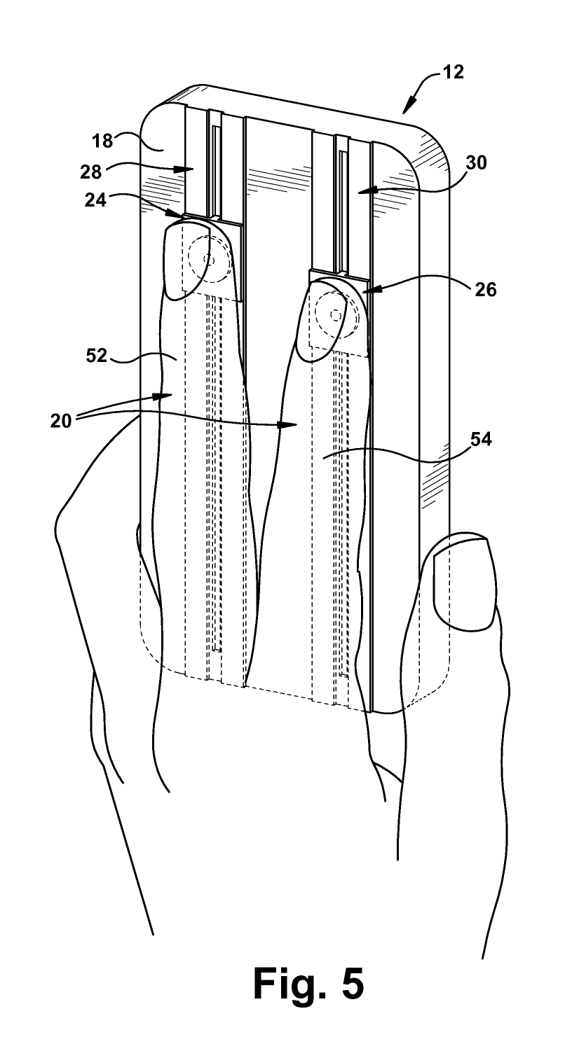

One aspect of the present invention includes a handheld, portable device 10 for providing biofeedback to a subject. The device 10 comprises a housing 12, at least one sensor 14, and a controller (not shown) that includes circuitry (not shown) and software (not shown). As shown in FIG. 1A, the device 10 includes a housing 12 that is ergonomically and aesthetically adapted for comfort and ease of use. The housing 12 has a generally rectangular shape defined by oppositely disposed front and back sides 16 and 18. The housing 12 can be made from one or a combination of durable materials, such as metals, metal alloys, plastics (e.g., hardened plastics), and various other known polymers. One skilled in the art will recognize that the shape of the housing 12 described and shown herein should not be limiting. For example, the housing 12 can take any form in which two sensors 14 are held between different digits (FIG. 5) of the hand of a subject so that two or more different physiological parameters can be measured. Although not shown in FIGS. 1A-B, it will be appreciated that the device 10 additionally includes at least one power source operably connected to the housing 12 (e.g., disposed within the housing). For example, the power source can include a single life or rechargeable battery.

The housing 12 also includes a user interface 22 that is operably connected thereto and competes with current smart phone applications for ease of use, convenience, and entertainment. As shown in FIG. 1A, for example, the user interface 22 is operably connected to the front side 16 of the housing 12. The user interface 22 can generally include any type of two-dimensional (2D) or three-dimensional (3D) display screen, such as an LCD screen with a resolution capable of displaying information and/or permitting information exchange between a subject and the device 10. For example, the user interface 22 can permit the graphical and/or textual exchange of information between the subject and the device 10.

The user interface 22 is dimensioned to optimize information exchange between the subject and the device 10. As shown in FIG. 1A, for example, the user interface 22 is sized to occupy substantially all of the front side 16. It will be appreciated that the user interface 22 can be smaller or larger than the one shown in FIG. 1A, and that the user interface can have any other desired shape (e.g., circular, ovoid, etc). Additionally, it will be appreciated that the device 10 can include more than one user interface 16. One example of the user interface 22 can include a graphical user interface (GUI), which allows a subject to interact with the device 10 in more ways than just typing. For example, a GUI can offer graphical icons and visual indicators, as opposed to text-based interfaces, typed command labels, or text navigation to fully represent information (e.g., physiological information and instructions) to a subject.

The device 10 additionally includes at least one sensor 14 configured to sense a physiological parameter of the subject. For example, the device 10 can include a plurality of fingertip sensors 14 for sensing different physiological parameters. Traditional biofeedback devices employ wet electrodes or sensors (i.e., sensors that require the application of a conductive gel or liquid in order to operate effectively) that are inaccurate and do not respond rapidly. Typically, such wet electrodes are connected to a separate electronics unit via wires. In contrast, the sensor 14 (or sensors) of the present invention includes a wire-free, dry electrode that eliminates the need for advance preparation with gels or liquids. This aspect of the present invention advantageously frees the subject from the limitations of wired connections, and allows the device 10 to be used with a heterogeneous mix of computing platforms. Further, the sensor 14 (or sensors) included as part of the device 10 is sensitive, accurate and rapidly responding, which eliminates or mitigates the interference of adjacent electrical activity and movement artifact(s).

The sensor 14 is capable of sensing any physiological parameter or characteristic associated with a subject and/or body organ function of the subject. Physiological parameters can be variable, meaning any physiological condition of a subject's body that may experience a measurable change. Physiological parameters can also be static, such as weight, height, etc. Physiological parameters can be indicative of, or associated with, ANS function. Non-limiting examples of physiological parameters that can be measured or obtained by the sensor 14 include electrocardiography data, pulse rate, blood pressure, respiration rate, skin temperature, surface electromyography data, electrocardiography data, skin conductance, digital peripheral temperature, blood volume pulse, and EEG data.

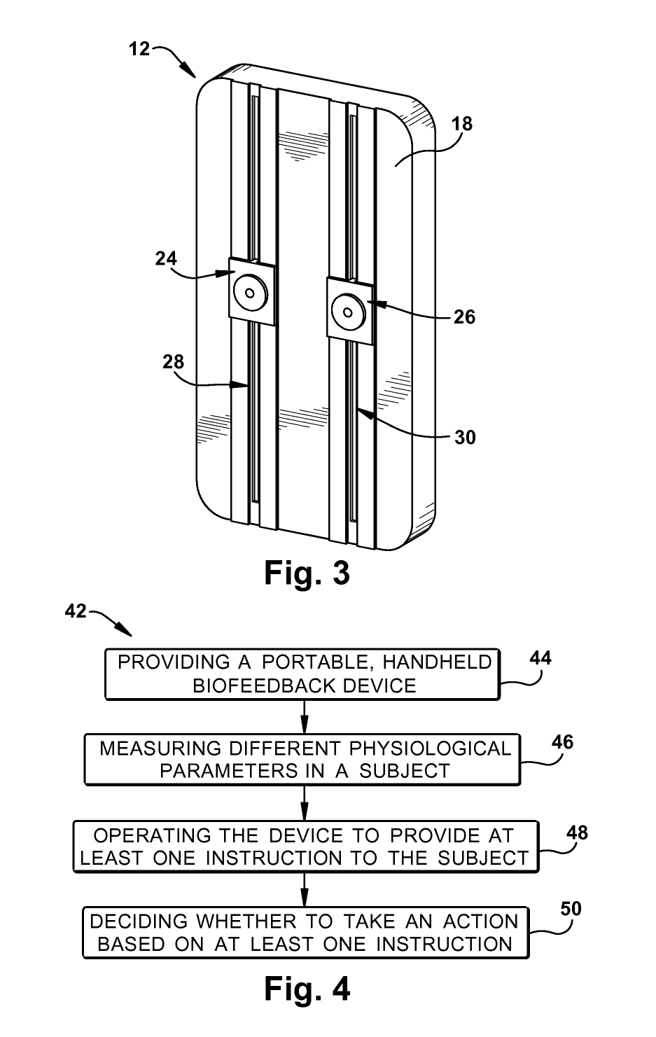

In one example of the present invention, the device 10 can include first and second fingertip sensors 24 and 26 that are configured to contact different digits 20 (FIG. 5) of a subject's hand. As shown in FIG. 1B, the first and second fingertip sensors 24 and 26 are operably mounted to the back side 18 of the housing 12. The first and second fingertip sensors 24 and 26 can be fixedly mounted to the housing 12 (FIG. 1B) or, alternatively, slidably mounted thereon via first and second tracks 28 and 30, respectively (FIG. 3). The first and second tracks 28 and 30 enable the first and second fingertip sensors 24 and 26 (respectively) to slide independent of one other along the back side 18 of the housing 12. The first and second tracks 28 and 30 account for different digit lengths and thereby allow the first and second fingertip sensors 24 and 26 to maintain contact with different digits 20 during use of the device 10 (FIG. 3).

The sensors 14, 24, and 26 (FIGS. 1A-B) can have any suitable 2-D or 3-D geometry to facilitate sensing of different physiological parameters. Although only first and second fingertip sensors 24 and 26 are shown in FIGS. 1A-3, it will be appreciated that the device 10 can include three or more sensors 14. Additionally, it will be appreciated that the sensor(s) 14 can be operably connected to any portion of the housing 12. Unlike biofeedback devices of the prior art, which typically include only one sensor for measuring physiological parameters, the device 10 of the present invention can advantageously include multiple sensors 14 that permit simultaneous and redundant sensing of different physiological parameters to improve robustness of multiple sensed parameters.

The device 10 of the present invention additionally includes a controller configured to provide biofeedback to the subject. The controller is in electrical communication with the sensor 14 (or sensors) and includes circuitry for collecting and storing physiological parameters. The ability of the device 10 to store data (e.g., physiological parameters) obtained from a subject is different from prior art devices and fosters the use of the device for not only research purposes, but also sharing data with a physician or coach. As used herein, the term “circuitry” can include electrical circuitry having at least one discrete electrical circuit, electrical circuitry having at least one integrated circuit, electrical circuitry having at least one application-specific integrated circuit, electrical circuitry forming a general purpose computing device configured by a computer program (e.g., a general purpose computer configured by a computer program that at least partially carries out processes described herein, or a microprocessor configured by a computer program that at least partially carries out processes described herein), electrical circuitry forming a memory device (e.g., forms of memory, such as random access, flash, read only, etc.), electrical circuitry forming a communications device (e.g., a modem, communications switch, optical-electrical equipment, etc.), and/or any non-electrical analog thereto, such as optical or other analogs. Those having skill in the art will recognize that the circuitry can be implemented in an analog fashion, a digital fashion, or some combination thereof.

Additionally, the controller includes software for providing biofeedback to a subject based on at least one collected and stored physiological parameter. The software can generally include one or more computer programs and related data that provide instructions to the circuitry. The software can comprise one or more known types of software, such as system software (e.g., an operating system), programming software (e.g., defining the syntax and semantics of various programs), and application software (e.g., end-user applications). Other examples of software can include firmware, device drivers, programming tools, and middleware. It will be appreciated that the software can additionally or optionally include at least one questionnaire to assess mental stress (e.g., how the subject feels stress).

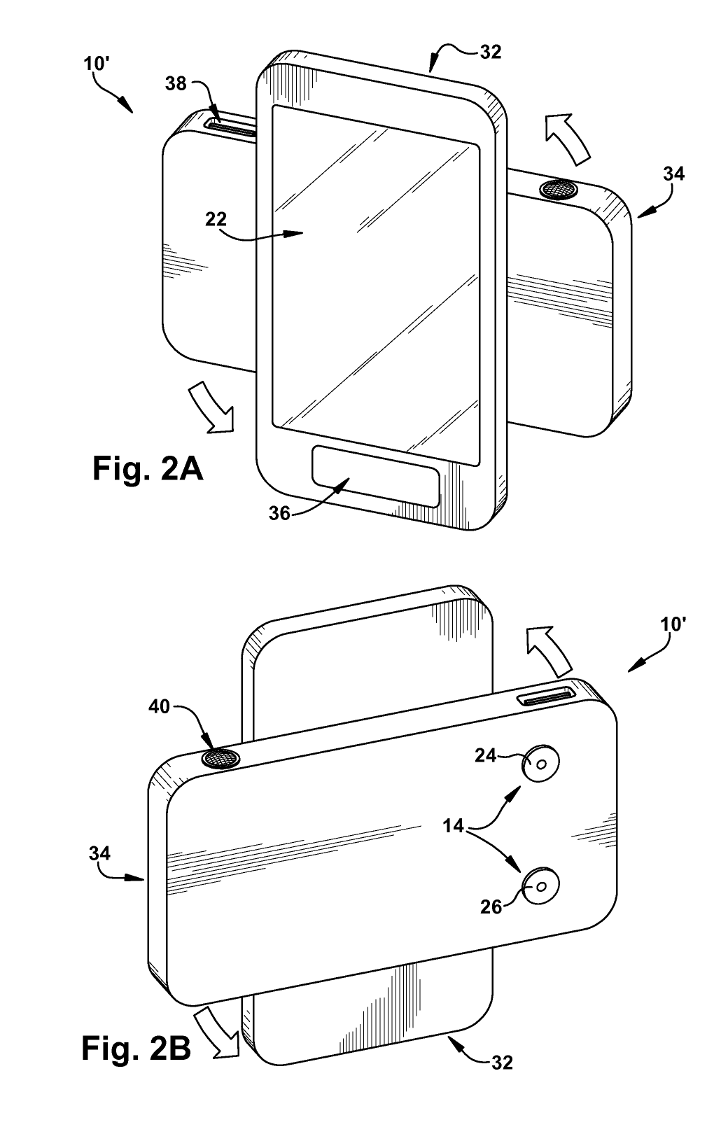

An alternative configuration of the device 10 is shown in FIGS. 2A-B. The device 10′ can include first and second housings 32 and 34 that are rotatable relative to one another. The first housing 32 can include a user interface 22 (as described above), and the second housing 34 can include a plurality of fingertip sensors 14 (as also described above), such as first and second fingertip sensors 24 and 26. The first and second housings 32 and 34 can be rotatably joined to one another via a joint (not shown), such as a ball-and-socket joint. The rotatable feature of the device 10′ allows a subject to contact different digits 20 with the fingertip sensors 14 (FIGS. 2A-B) while simultaneously viewing and/or manipulating the user interface 22 without losing contact between the digits and the fingertip sensors. This, in turn, ensures that the physiological parameters are continuously and reliably recorded during operation of the device 10′.

It will be appreciated that the device 10 and 10′ of the present invention can include one or more optional components. As shown in FIGS. 1A-B, for example, the device 10 and 10′ can include a control interface 36 (e.g., a keyboard) that allows the subject or a third party (e.g., a medical practitioner) to directly enter data (e.g., age, height, weight, etc.) into the device. The device 10 and 10′ can also include one or more input/output (I/O) ports 38. The I/O ports 38 can be used, for example, to transfer data into and/or out of the device 10 and 10′ or for connection to a power source. Additionally, the device 10 and 10′ can include at least one speaker 40 for providing auditory signals and/or instructions to a subject.

The device 10 and 10′ can additionally or optionally be configured to enable remote monitoring and/or data storage capabilities. For example, the device 10 and 10′ can include a communications interface (not shown) for transmitting and/or receiving data between the device and at least one remote device (not shown). Remote devices can include any device capable of connecting to and communicating with the device 10 and 10′. Examples of remote devices can include, but are not limited to, desktop computers, mp3 players, mobile phones, PDAs, game consoles, and set-top boxes. Various wire-based protocols, such as USB, Ethernet, and FireWire, and wireless protocols, such as Bluetooth and Wi-Fi, can be used to facilitate communication between the device 10 and 10′ and a remote device. In addition, it will be appreciated that various proprietary protocols can be developed for communicating between the device 10 and 10′ and a remote device.

In one example of the present invention, a device (not shown) similar to the one in FIGS. 1A-3 can be configured as a PC version for the home or workplace. In this configuration, the device can include three or more sensors 14 so that additional physiological parameters can be monitored, as well as a wireless component (e.g., a watch) (not shown) that can be worn by the subject when he or she is not close to the workstation. The wireless component can monitor one or more physiological parameters and send a signal to the subject when one or more of the physiological parameters is not within an optimal range. As discussed above, this configuration of the present invention allows the subject to optimize physiology privately while engaged in routine activity (e.g., in the home or workplace) or in high demand/performance situations.

Another aspect of the present invention includes a method 42 (FIG. 4) for self-regulating at least one physiological state of a subject. As discussed above, one aspect of the present invention includes a portable, handheld biofeedback device 10 and 10′ that provides multi-parameter feedback and guidance on managing physiological states. Advantageously, the method 42 of the present invention provides physiological monitoring and biofeedback training to enable self-regulation for a variety of physiological states, including personal health and wellness, disease management, and performance enhancement. Although the method 42 is described below primarily in terms of stress management, it will be appreciated that the method can find utility in managing a variety of other diseases, such as those that include significant involvement of the ANS.

Referring to FIG. 4, one step of the method 42 includes providing a portable, handheld biofeedback device 10 at Step 44. As discussed above, the biofeedback device 10 can generally comprise a housing 12 having a user interface 22 operably connected thereto, at least one sensor 14 operably connected to the housing, and a controller in communication with the sensor(s) to provide biofeedback to a subject. In one example of the present invention, the biofeedback device 10 can have a configuration similar to the one shown in FIGS. 1A and 3; that is, the device can include a single user interface 22, at least one I/O port 38, a speaker 40, and first and second fingertip sensors 24 and 26 slidably connected to first and second tracks 28 and 30 (respectively).

The device 10 is used to measure at least one physiological parameter. At Step 46, for example, the device 10 can be used to measure at least two different physiological parameters and/or a particular pattern or trend for one or more physiological parameters (i.e., rather than a single physiological parameter). Current biofeedback programs, although they can monitor many physiological processes simultaneously, focus on training a subject to control one physiological parameter at a time. The present invention capitalizes on the fact that each subject has a unique physiological response pattern, and if the entire pattern is shifted, then positive outcomes can ensue more quickly. The method 42 of the present invention can identify an individual response pattern (or patterns) and then feedback the pattern to the subject, thereby training the subject to control his or her general arousal level rather than a specific physiological manifestation. Moreover, identification of a specific pattern of arousal, which is optimal for performance, is a far more powerful approach for biofeedback training than conventional biofeedback devices.

Different physiological parameters are measured by contacting at least a portion of the subject with the sensor 14 (or sensors). As shown in FIG. 5, for example, the first and second digits 52 and 54 of the subject are contacted with the first and second fingertip sensors 24 and 26, respectively, by placing the fingertips of the digits onto the sensors. Prior to measuring the physiological parameters, the biofeedback device 10 can be calibrated to provide a baseline or standard measurement or one or more physiological parameters (or pattern of physiological parameters). As described in more detail below, the baseline or standard measurement can then be used (e.g., by the software) to provide instruction(s) for regulating a physiological state in the subject.

The device 10 can be calibrated using one or a combination of techniques. For example, the device 10 may include a calibration function (e.g., as part of the software) that enables the subject to provide standardized information to the device, such as the way the subject performs/achieves particular activities, certain body positions/orientations, and/or body status information (e.g., physiological parameters). Such information can be directly provided by the subject to the device (e.g., via the user interface 22 or the control interface 36) or through an external device (e.g., via an I/O port 38). The information can include data that was previously obtained under training or standardized conditions. In one example of the present invention, the information used to calibrate the device can include data that was previously obtained during a stressful situation. While the subject is subjected to stress, the device 10 can sense and record physiological data that will allow the device to reliably recognize similar activities and/or body status during subsequent use.

Along with physiological parameters that the device 10 actually “observes” (i.e., senses and records) during normal activity, the data captured during calibration of the device may be used to build a database (e.g., stored on the device) of physiological parameters, body positions/orientations, body status, etc. that is correlated to sensed data so that particular activities and/or sequences of activities may be quickly and reliably identified. For example, the physiological parameters, status, and/or location of a subject may be logged throughout a full day or over a period of weeks or years.

After calibrating the device 10 and recording and storing a certain physiological parameter or parameters, at least one biofeedback score is calculated by the device. The biofeedback score is based on at least one of the collected and recorded physiological parameters, and may be further based on a previously trained technique that is known to the subject as a means for regulating the particular physiological state. For example, the biofeedback score may be indicative of increased or decreased ANS function, such as increased or decrease heart rate. It will be appreciated that the biofeedback score can additionally and/or optionally be based on a certain physiological parameter (or parameters) that is related to a particular psychological state (e.g., stress). Such psychological parameters can be obtained from a subject-responsive means, such as a questionnaire that is included as part of the software comprising the device 10. Thus, the biofeedback score can be a “two-dimensional” indicator of a particular physiological state by combining aspects of various physiological and psychological parameters.

Based on the biofeedback score, the device 10 can generate at least one instruction at Step 48. The instruction can be in the form of a graphical indication (e.g., displayed on the user interface), an auditory noise or voice command, and/or one or more vibrations. The instruction can include directions to the subject to modulate a behavior (or behaviors) if one or more of the sensed physiological parameters is not in an optimal range.

At Step 50, the subject decides whether to take one or more actions in response to the instruction(s) and thereby self-regulate one or more physiological states. Where the instruction informs the subject that he or she is no longer in an optimal zone for a particular pattern or physiological parameter, for example, the subject can decide to modify one or more behaviors (e.g., breathing or physical exertion) to bring the pattern or physiological parameter back into an optimal zone. Alternatively, the instruction may inform the subject that he or she is currently in an optimal zone. In this case, the subject may decide not to take any action(s) to maintain a particular physiological state.

In one example of the present invention, the subject can mitigate or prevent a disease or condition associated with stress by self-regulating at least one pattern or physiological parameter associated with stress. To do so, the device 10 can be calibrated to a particular subject so that the subject is taught to recognize an ANS and EEG pattern associated with optimal performance (e.g., less stress). For example, EEG waves and ANS patterns can be monitored during simulated stressful situations and then used to calibrate the device 10. The subject can then be taught how to maintain an optimal state of EEG waves and ANS patterns to optimally reduce stress. Once the device 10 has been calibrated, a brief signal (e.g., a vibration or noise) can indicate that the subject is no longer in the optimal zone during use of the device. The subject can then adjust his or her breathing, for example, as the subject has previously learned during training.

In another example of the present invention, a performance characteristic of a subject can be self-regulated. A performance characteristic can include any physiological function (e.g., brain function, heart function, etc.) that needs to be optimized for a given task undertaken by the subject. Certain individuals, such as pilots, professional athletes, and soldiers require optimal physiological functioning due to the high demands of their profession. The device 10 can function to provide subjects with biofeedback who need precise physiological function in a narrow window, with no room for error, in real time. For such individuals, the device 10 can be calibrated in a high stress environment that mimics the stress that the subject will endure during a given task. The subject can then be trained how to optimally respond to the stress. When the subject is subsequently operating within his or her given profession, the device 10 can provide instruction(s) so that the subject can optimize one or more performance characteristics to perform at an optimal level. It should be appreciated that the device 10 can also be used to improve not only the physical performance characteristics of a subject, but also the cognitive performance characteristics of a subject.

It will also be appreciated that all or only a portion of the method 42 can be performed remotely. By incorporating a communications interface into the device 10, for example, a subject could report to a central monitor (e.g., his or her physician or personal trainer) on a regular basis (e.g., daily or weekly) to demonstrate progress in regulating a particular physiological state and, thus, provide an indication of when the subject is having difficulty. In this case, the potential for taking action prior to the development of a larger problem can be mitigated or prevented.

From the above description of the invention, those skilled in the art will perceive improvements, changes, and modifications. For example, it will be appreciated that biofeedback provided by the present invention can include psychological indicators (e.g., perceived stress, life stress, coping style, personality factors, etc.) that may additionally or optionally serve as a basis for self-regulating at least one physiological state in a subject. Such improvements, changes, and modifications are within the skill of one in the art and are intended to be covered by the appended claims.

Having described the invention, the following is claimed:

1. A handheld, portable device for providing biofeedback to a subject, said device comprising:
a housing having a user interface operably connected thereto;
at least one sensor configured to sense a physiological parameter, said at least one sensor being operably connected to said housing; and
a controller configured to provide biofeedback to the subject, said controller being in electrical communication with said at least one sensor.

2. The device of claim 1, further comprising first and second fingertip sensors operably connected to said housing, each of said first and second fingertip sensors configured to sense a different physiological parameter.

3. The device of claim 1, wherein said controller further comprises:
circuitry for collecting and storing the physiological parameter, said circuitry being in communication with said at least one sensor; and
software for providing the biofeedback to the subject based on the collected and stored physiological parameter.

4. The device of claim 1, wherein the physiological parameter is indicative of autonomic nervous system (ANS) function or other physiological function.

5. The device of claim 2, wherein said first and second fingertip sensors measure at least one of surface electromyography, electrocardiography, skin conductance, digital peripheral temperature, blood volume pulse, respiration, electroencephalography, and blood pressure.

6. The device of claim 2, wherein said first and second fingertip sensors eliminate interference caused by adjacent electrical activity and movement artifact.

7. The device of claim 1 further comprising a communications interface for transmitting and/or receiving data between said device and at least one remote device in communication with said device.

8. The device of claim 1, wherein the biofeedback is visually displayed on said user interface.

9. The device of claim 1, wherein the biofeedback is auditory.

10. The device of claim 1, wherein the biofeedback is provided to the subject in substantially real time.

11. A method for self-regulating at least one physiological state of a subject, said method comprising the steps of:
providing a portable, handheld device comprising a housing having a user interface operably connected thereto, at least one sensor operably connected to the housing, and a controller in electrical communication with the at least one sensor;
operating the device to provide at least one instruction to the subject based on at least one collected and recorded physiological parameter; and
deciding whether to take an action in response to the at least one instruction to regulate the at least one physiological state.

12. The method of claim 11, wherein said step of providing a portable, handheld device further includes providing a housing having first and second fingertip sensors operably connected thereto.

13. The method of claim 12, wherein said step of contacting the first and second fingertip sensors with the subject further comprises contacting first and second fingertips of the subject with the first and second fingertip sensors, respectively.

14. The method of claim 11, wherein said step of operating the device further comprises the steps of:
calibrating the device; and
calculating at least one biofeedback score based on the at least collected and recorded physiological parameter.

15. The method of claim 14, wherein said step of calibrating the device further comprises inputting at least one control physiological parameter into the device.

16. The method of claim 14, wherein said step of calculating at least one biofeedback score further comprises determining at least one physiological response pattern based on the at least one collected and recorded physiological parameter.

17. The method of claim 11, wherein self-regulating the at least one physiological state mitigates or prevents a disease associated with ANS or other physiological dysfunction in the subject.

18. The method of claim 11, wherein self-regulating the at least one physiological state mitigates or prevents a disease associated with stress in the subject.

19. The method of claim 11, wherein self-regulating the at least one physiological state improves a performance characteristic in the subject.

20. The method of claim 18, further comprising:
calibrating the device; and
calculating at least one stress score based on the at least one collected and recorded physiological parameter.

21. The method of claim 20, wherein said step of calculating at least one stress score further comprises determining at least one stress response pattern based on at least one of the collected and sensed physiological parameters.

22. The method of claim 21, further comprising changing an arousal level of the subject based on the at least one stress response pattern to mitigate or prevent stress.
